(19) United States
(12) Patent Application Publication
(10) Pub. No.: US 2013/0345234 A1
(43) Pub. Date: Dec. 26, 2013

(54) FGFR AND LIGANDS THEREOF AS BIOMARKERS FOR BREAST CANCER IN HR POSITIVE SUBJECTS

(71) Applicants: Humphrey Athelstan Roy Gardner, Salem, MA (US); Michael Shi, Chalfont, PA (US); Alejandro Yovine, Folgensbourg (FR)

(72) Inventors: Humphrey Athelstan Roy Gardner, Salem, MA (US); Michael Shi, Chalfont, PA (US); Alejandro Yovine, Folgensbourg (FR)

(21) Appl. No.: 14/003,312

(22) PCT Filed: Mar. 15, 2012
(86) PCT No.: PCT/US12/29205

§ 371 Date: Sep. 5, 2013

Related U.S. Application Data
(60) Provisional application No. 61/453,723, filed on Mar. 17, 2011.

(51) Int. Cl. C12Q 1/68

(52) U.S. Cl. CPC C12Q 1/6886
USPC 514/253.07; 435/6.11

(57) ABSTRACT

The present invention describes methods for diagnosing, treating and determining the prognosis of breast cancer HR+ patient, the methods including detecting the amplification of one or more biomarkers comprising a FGFR ligand such as FGF3, FGF4, FGF19, and/or a FGFR, such as for example FGFR1 in a subject; determining an FGFR1 inhibitor for treating the subject based on the amplification of the one or more biomarkers in the subject; administering to the subject in need thereof the FGFR1 inhibitor and using the one or more biomarkers to indicate prognosis of the subject treated with the FGFR1 inhibitor.

### FIELD OF THE INVENTION

The present invention relates to diagnosing and determining the prognosis of cancer patients using a biomarker. In particular, the present invention is directed to diagnosing, treating and determining the prognosis of breast cancer patients using a biomarker based on a certain fibroblast growth factors (FGF) ligand loci, for example FGF3, FGF4 and FGF19 and combinations of FGF3 with FGF4, FGF19 and a certain fibroblast growth factor receptor (FGFR), FGFR1.

### BACKGROUND OF THE INVENTION

Therapeutic options for the treatment of breast cancers include surgery, radiotherapy, endocrine therapy, and cytotoxic chemotherapy. Limited attempts to use molecular markers that can provide prognostic information and/or predict treatment outcome have been recently disclosed.

U.S. Pat. Appl. Publ. No. 2007/0218512 A1 discloses a biomarker based on a certain human matrix metalloproteinase (MMP), MMP-26 for diagnosing and determining prognosis of breast cancers associated with the hormone-based estrogen receptor (ER). The presence of MMP-26 in a subject is most favorable in early-stage breast cancer. When a subject has early stage breast cancer, the prognosis is generally considered good when MMP-26 is present. However, it is further disclosed that other factors can also be taken into consideration when making this assessment, such as clinical information and the presence or absence, and expression levels, of other biomarkers. Therefore, the need remains for a reliable test for diagnosing and determining prognosis of breast cancer using specific therapeutic agents. Such a test for diagnosing and determining prognosis of breast cancer would not require consideration of the presence or absence, and expression levels, of other biomarkers.

Fibroblast growth factors (FGFs) and their receptors (FGFR) are a highly conserved group of proteins with instrumental roles in angiogenesis, vasculogenesis, and wound healing, as well as tissue patterning and limb formation in embryonic development. FGFs and FGFRs affect cell migration, proliferation, and survival, providing wide-ranging impacts on health and disease.

The FGFR family comprises four major types of receptors, FGFR1, FGFR2, FGFR3, and FGFR4. These receptors are transmembrane proteins having an extracellular domain, a transmembrane domain, and an intracytoplasmic domain. Each of the extracellular domains contains either two or three immunoglobulin (Ig) domains. Some FGFRs exist in different isoforms which differ in specific segments of the molecule, such as FGFR-IIIb and FGFR1-IIIc, which differ in the C-terminal region of the third Ig domain. Transmembrane FGFRs are monomeric tyrosine kinase receptors, activated by dimerization, which occurs at the cell surface in a complex of FGFR dimers, FGF ligands, and heparin glycans or proteoglycans. Extracellular FGFR activation by FGF ligand binding to an FGFR initiates a cascade of signaling events inside the cell, beginning with the receptor tyrosine kinase activity.

FGFR1 amplification was observed in 8.7% of the tumors and was significantly more prevalent in patients greater than 50 years of age and in tumors that lacked HER2 expression. Studies have demonstrated that FGFR1 gene amplification correlates with FGF oncogene expression. FGFR1 activity is required for the survival of a FGFR1 amplified breast cancer cell line as described by Reis-Filho J S, Simpson P T, Turner N C, Lambros M B, Jones C et al., in the publication Clin. Cancer Res 12, 6652-6662 (2006). FGFR1 amplification is uncommon in HER2 amplified breast cancer, as described by Elbauomy Elsheikh S, Green A R, Lambros M B, Turner N C, Grainge M J et al., in the publication Breast Cancer Res 9, R23 (2007), suggesting that amplification of HER2 and FGFR1 may be alternative and mutually exclusive mechanisms of activating similar downstream pathways that drive tumor proliferation and poor prognosis. The same authors suggested that FGFR1 amplifications were associated with poor prognosis in patient with estrogen receptor (ER) positive tumor. High level amplification of FGFR1 is predominantly found in ER positive, luminal B type, poor prognosis breast cancers as described by Chin K, DeVries S, Fridly and J, Spellman P T, Roydasgupta R et al., in the publication Cancer Cell 10, 529-541 (2006), as described by Courjal F, Theillet C, (1997). Comparative genomic hybridization analysis of breast tumors with predetermined profiles of DNA amplification is presented in Cancer Res 57, 4368-4377 and also described by Reis-Filho J S, Simpson P T, Turner N C, Lambros M B, Jones C et al., in the publication Clin. Cancer Res 12, 6652-6662 (2006).

It was surprisingly discovered that receptor amplification of FGFR1 and ligand amplification of FGF3 are useful as molecular markers for breast cancers treated by an FGFR inhibitor (FGFR inhibitor sensitive breast cancers) and provide a reliable method for both diagnosing and determining prognosis in patients undergoing treatment for breast cancer using an FGFR inhibitor.

### SUMMARY OF THE INVENTION

The present invention also provides a method for diagnosing cancer associated with FGF ligand amplification or FGFR amplification in a subject, the method comprising the step of: detecting amplification of a biomarker comprising a FGF ligand in the subject, wherein the presence or amounts of the FGF ligand is indicative of the cancer.

The present invention also provides a method for diagnosing cancer associated with FGF3 amplification in a subject, the method comprising the step of: detecting amplification of one or more biomarkers selected from FGF3, FGF4, FGF19, FGFR1 and combinations thereof in the subject, wherein the amplification of the one or more biomarkers is indicative of the cancer.

The present invention also provides a method for diagnosing cancer associated with FGFR1 amplification in a subject, the method comprising the step of: detecting amplification of one or more biomarkers selected from FGF3, FGF4, FGF19, FGFR1 and combinations thereof in the subject, wherein the amplification of the one or more biomarkers is indicative of the cancer.

The present invention also provides a method of treating cancer in a subject, the method comprising the steps of: (a) detecting the amplification of one or more biomarkers selected from FGF3, FGF4, FGF19, FGFR1 and combinations thereof in a subject; and (b) determining an FGFR1 inhibitor for treating the subject based on the amplification of the one or more biomarkers in the subject; and administering to the subject in need thereof the FGFR inhibitor.

The present invention also provides a method for determining the prognosis of a subject having cancer and treated with an FGFR1 inhibitor, the method comprising the step of: detecting one or more biomarkers selected from a FGFR ligand, e.g. FGF3, FGF4, FGF19, a FGF R, e.g. FGFR1, FGFR2 and combinations thereof in the subject, wherein the presence or amounts of the one or more biomarkers is indicative of the prognosis of the subject treated with the FGFR1 inhibitor.

The present invention also provides a method for determining the prognosis of a subject having cancer and treated with an FGFR1 inhibitor, the method comprising the step of: detecting a biomarker comprising FGF3 in the subject, wherein the presence or amounts of FGF3 is indicative of the prognosis of the subject and treated with the FGFR1 inhibitor.

The present invention also provides a kit comprising an assay for determining the presence or amounts of FGF3 in a subject.

The present invention also provides a kit comprising an assay for determining the presence or amounts of one or more FGF3, FGF4, FGF19, FGFR1 and combinations thereof in a subject.

### DETAILED DESCRIPTION OF THE INVENTION

As used herein, subject includes, but is not limited to, any mammal (e.g., a human, horse, pig, rabbit, dog, sheep, goat, non-human primate, cow, cat, guinea pig or rodent). The term does not denote a particular age or sex. Thus, adult and newborn subjects, as well as fetuses, whether male or female, are intended to be covered. A patient refers to a subject afflicted with a disease or disorder. The term patient includes human and veterinary subjects.

As used herein, treatment means medical management of a patient with the intent to cure, ameliorate, stabilize, or prevent a cancer. This term includes active treatment that is directed toward removal of the cause of the associated cancer.

The term diagnosis of a cancer refers to detecting amounts of one or more biomarkers selected from FGF3, FGF4, FGF19, FGFR1 and combinations thereof in the subject, wherein the presence or amounts of the one or more biomarkers is indicative of the cancer. The term prognosis encompasses predictions about the likely course of cancer or cancer progression, particularly with respect to likelihood of remission, relapse, tumor recurrence, metastasis, and death. Good prognosis refers to the likelihood that a patient afflicted with cancer, particularly breast cancer, will remain cancer-free. Poor prognosis is intended to mean the likelihood of a relapse or recurrence of the underlying cancer or tumor, metastasis, or death.

Methods for detecting amplification, such as locus amplification of a FGFR ligand or a FGFR such as for example FGF3, FGF4, FGF19 and FGFR1 are methods such as in situ chromosome hybridization. The person skilled in the art would recognize which are the methods of in-situ hydridization that allow the detection an quantification of locus amplification. Such methods are for example CISH, SISH or q-PCR. Such methods are well known in the art and include but are not limited to western blots, northern blots, southern blots, ELISA, immunoprecipitation, immunofluorescence, flow cytometry, immunohistochemistry, nucleic acid hybridization techniques, nucleic acid reverse transcription methods, and nucleic acid amplification methods. The term “detecting amplification” is intended to mean determining the presence and quantity of a biomarker gene or protein. In order to determine amplification, the sample to be examined may be compared with a corresponding sample that originates from a healthy person, and the number of copy of the locus is superior in the sample than in the sample originated from the healthy person, or the number of copy of the locus is superior to one, for example the locus is amplified 3, 5, 6, 7, 8, 9, 10 or greater than 10 times.

FGF3, FGF4, FGF19 and FGFR1 biomarkers of the invention are obtained from samples from a subject. Examples of such samples include but are not limited to blood, lymph, urine, gynecological fluids, biopsies, and smears. Bodily fluids useful in the present invention include blood, urine, saliva, nipple aspirates, or any other bodily secretion or derivative thereof. Blood can include whole blood, plasma, serum, or any derivative of blood. In exemplary embodiments, the sample comprises breast cells, including breast tissue from a biopsy or a breast tumor tissue sample. However, the sample need not comprise breast tissue, and can be obtained from normal tissue, fluid, or cells. Samples may be obtained from a subject by a variety of techniques including, for example, by scraping or swabbing an area, by using a needle to aspirate bodily fluids, or by removing a tissue sample (i.e., biopsy). Methods for collecting various samples are well known in the art.

Cancers diagnosed using one or more biomarkers of the invention include, for example, leukemia, including B-cell acute lymphoblastic leukemia, chronic myelomonocytic leukemia, chronic lymphocytic leukemia, and chronic myeloid leukemia; in lymphoma, including Hodgkin's lymphoma, non-Hodgkin's lymphoma, and extranodal lymphoma; in myeloma, including plasmacytoma; in sarcoma, including malignant neoplasms of the bone and soft tissues; in neurologic cancer, including malignant neoplasms of the brain; in breast cancer, including malignant neoplasms of the female breast; in digestive tract/gastrointestinal cancer, including malignant neoplasms of the ampulla of Vater, appendix, colon, duodenum, esophagus, liver, pancreas, peritoneum, rectum, small intestine, and stomach; in endocrine cancer, including malignant neoplasms of the adrenal gland, islets of Langerhans, and thyroid gland; in eye cancer, including malignant neoplasms of the eye; in genitourinary cancer, including malignant neoplasms of the bladder, kidney, prostate, and testis; in gynecologic cancer, including malignant neoplasms of the uterine cervix, myometrium, ovary, uterus, endometrium, placenta, and vulva; in head and neck cancer, including malignant neoplasms of the larynx, salivary gland, nasal cavity, oral cavity, parotid gland, and tongue; in respiratory/thoracic cancer, including malignant neoplasms of the lung, thymus, and trachea; and in skin cancer.

According to one embodiment, a method is provided for diagnosing breast cancer associated with a FGFR ligand amplification in a subject, the method comprising the step of: detecting amplification of one or more biomarkers selected from FGF3, FGF4, FGF19, FGFR1 and combinations thereof in the subject, wherein the amplification of the one or more biomarkers is indicative of the cancer.

According to a separate embodiment, a method is provided for diagnosing breast cancer associated with HR+, and a FGFR amplification and/or FGFR ligand amplification in a subject, the method comprising the step of: detecting amplification of one or more biomarkers selected from FGF3, FGF4, FGF19, FGFR1 and combinations thereof in the subject, wherein the presence and amplification of the one or more biomarkers is indicative of the cancer.

According to another embodiment, a method is provided for prognostic of breast cancer associated with HR+, and a FGFR amplification and/or FGFR ligand amplification in a subject, the method comprising the step of: detecting amplification of one or more biomarkers selected from FGF3, FGF4, FGF19, FGFR1 and combinations thereof in the subject, wherein the presence and amplification of the one or more biomarkers is indicative of the breast cancer disease responsiveness to the treatment of the breast cancer with a FGFR1 inhibitor, such as for example dovitinib or a tautomer, or a pharmaceutically acceptable salt thereof.

According to one embodiment, an FGFR inhibitor, 4-amino-5-fluoro-3-[6-(4-methylpiperazin-1-yl)-1H-benzimidazol-2-yl]-1H-quinolin-2-one or a tautomer thereof, is useful in the treatment of breast cancer and other tumor types with FGFR and FGF pathway activations, as determined by a FGFR1 or FGF3 biomarker.

The compound 4-amino-5-fluoro-3-[6-(4-methylpiperazin-1-yl)-1H-benzimidazol-2-yl]-1H-quinolin-2-one (also referred to as dovitinib) or a tautomer thereof or a pharmaceutically acceptable salt thereof, of formula I

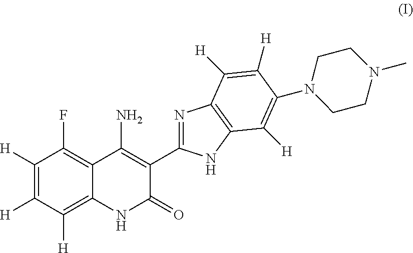 [SMILES: CN1CCN(c2ccc3nc(-c4c(N)c5c(F)cccc5[nH]c4=O)[nH]c3c2)CC1]

inhibits certain protein kinases, such as tyrosine receptor kinases (RTKs). The compound and its pharmaceutically acceptable salts, including the mono-lactic acid salt, are described in U.S. Pat. Nos. 6,605,617, 6,774,237, 7,335,774, and 7,470,709, and in U.S. patent application Ser. Nos. 10/982,757, 10/982,543, and 10/706,328, and in the published PCT applications WO 2006/127926 and WO2009/115562.

The antitumor activity of dovitinib was evaluated in a variety of tumor xenograft models in athymic mice. In all models tested, dovitinib administered orally resulted in anti-tumor responses, ranging from growth inhibition and stasis to regression in tumor models driven by activating mutations of dovitinib targets. Furthermore, in a model of disseminated disease, colonization of breast cancer cells in the livers of mice from a subcutaneous (SC) xenograft primary was significantly reduced by oral treatment using dovitinib. To demonstrate that direct inhibition of RTKs in tumors was a primary mechanism of xenograft growth inhibition by dovitinib, tyrosine phosphorylation of FGFR downstream-signaling proteins AKT (also known as protein kinase B) and ERK (also known as MAPK) was shown to be inhibited following a single oral dose of dovitinib. Inhibition was observed up to 24 hours in certain models.

According to one embodiment, a method of treating breast cancer in a subject, the method comprising the steps of: (a) detecting the presence or amount of one or more biomarkers selected from FGF3, FGF4, FGF19, FGFR1 and combinations thereof in a subject; and (b) determining an FGFR1 inhibitor for treating the subject based on the presence or amount of the one or more biomarkers in the subject; and administering to the subject in need thereof the FGFR1 inhibitor.

According to one embodiment, a method for determining the prognosis of a subject having cancer and treated with an FGFR1 inhibitor, the method comprising the step of: detecting one or more biomarkers selected from FGF3, FGF4, FGF19, FGFR1 and combinations thereof in the subject, wherein the presence or amounts of the one or more biomarkers is indicative of the prognosis of the subject treated with the FGFR1 inhibitor.

FGFR1 inhibitor, such as dovitinib or a tautomer thereof, or a pharmaceutically acceptable salt thereof, for use in the treatment or prognostic of breast cancer wherein the breast cancer is HR+ and wherein the patient has locus amplification of one or more FGFR ligand, such as FGF3, FGF4, FGF19, and/or FGFR, such as FGFR1 or FGFR2.

Kits for practicing the methods disclosed herein are further provided. By “kit” is intended any manufacture (e.g., a package or a container) comprising at least one reagent for specifically detecting the expression of one or more of FGF3, FGF4, FGF19 and FGFR1. The kit can be promoted, distributed, or sold as a unit for performing the methods of the present invention. Additionally, the kits can contain a package insert describing the kit and methods for its use. Any or all of the kit reagents may be provided within containers that protect them from the external environment, such as in sealed containers. Positive and/or negative controls can be included in the kits to validate the activity and correct usage of reagents employed in accordance with the invention. Controls may include samples, such as tissue sections, cells fixed on glass slides, etc., known to be either positive or negative for the presence of the biomarker of interest.

Specific embodiments of the invention will now be demonstrated by reference to the following examples. It should be understood that these examples are disclosed solely by way of illustrating the invention and should not be taken in any way to limit the scope of the present invention.

### EXAMPLE 1

Clinical Study to Test Efficacy of Dovitinib in FGFR1 Amplified and Non-Amplified Metastatic Breast Cancer

A multicenter, open-label phase 2 trial of dovitinib was conducted to evaluate the clinical activity of dovitinib and to test the clinical efficacy in FGFR1 amplified and non-amplified metastatic breast cancer. The efficacy and safety of dovitinib was studied in 4 groups of metastatic breast cancer patients: (Group 1: FGFR1+, HR+), (Group 2: FGFR1+, HR−) (Group 3: FGFR1−, HR+), (Group 4: FGFR1−, HR−). Patient selection was performed according to FISH/CISH for FGFR1 (cut-off≧6 gene copies). Dovitinib (500 mg) was administered once daily on a 5-day on/2-day off schedule. The primary endpoint was RECIST best overall response rate in pts with measurable disease per external radiology review.

Inclusion Criteria:
1. Patients have histological confirmation of breast carcinoma with a clinical diagnosis of IBC based on presence of inflammatory changes in the involved breast, including diffuse erythema and edema (peau d orange), with or without an underlying palpable mass involving the majority of the skin of the breast. Pathological evidence of dermal lymphatic invasion should be noted but is not required for diagnosis.
2. Patients have stage IV disease with local or distant relapse
3. Patients have negative HER2 expression by IHC (defined as 0 or 1+), or FISH (fluorescence in situ hybridization). If HER2 is 2+, negative HER2 expression must be confirmed by FISH.
4. Patients are able to swallow and retain oral medication.
5. Patients have ECOG performance status 0-2.
6. Patients have received up to 3 standard chemotherapies for metastatic disease and have relapsed.
7. Patients have ability and willingness to sign written informed consent.
8. Patients are 18 years of age or older.
9. Female patients of childbearing potential (A female not free from menses>2 years or not surgically sterilized) must be willing to use two adequate barrier methods of contraception to prevent pregnancy or agree to abstain from heterosexual activity throughout the study.
10. Female patients of childbearing potential must have negative serum pregnancy test.
11. If Patients have been treated with anti-VEGF agents, such as Bevacizumab, last dose must be >4 weeks.
12. Patients have biopsy tissue of the metastatic disease (including chest wall or regional nodes) available (paraffin blocks or up to 20 unstained slides), if no biopsy tissue available, a biopsy (or thoracentesis if patient has pleural effusion only) of the metastatic disease will be performed to confirm the diagnoses.

Exclusion Criteria:
1. Patients are receiving concurrent anti-cancer therapy (chemotherapy, immunotherapy, radiation therapy and biological therapy) while taking study medication.
2. Abnormal liver functions consisting of any of the following: a) Serum bilirubin>/=1.5×ULN, b) AST and ALT>/=2.5×ULN, (for patients with known liver metastasis, AST and ALT</=5×ULN is allowed), ANC<1.5.
3. Patients have an active infection and require IV or oral antibiotics.
4. Impaired cardiac function or clinically significant cardiac diseases, including any of the following: a) History or presence of serious uncontrolled ventricular arrhythmias or presence of atrial fibrillation; b) Clinically significant resting bradycardia (<50 beats per minute); c) LVEF<45% assessed by 2-D echocardiogram (ECHO) or Multiple gated acquisition scanning (MUGA); d) Any of the following within 6 months prior to study entry: myocardial infarction (MI), severe/unstable angina, Coronary Artery Bypass Graft (CABG), Congestive Heart Failure (CHF), Cerebrovascular Accident (CVA), Transient Ischemic Attack (TIA), Pulmonary Embolism (PE); e) Uncontrolled hypertension defined by an SBP>150 and/or a DBP>100 mm Hg with or without anti-hypertensive medication.
5. History of gastrointestinal disorders (medical disorders or extensive surgery) which may interfere with the absorption of the study drug.
6. Patients have a concurrent disease or condition that would make them inappropriate for study participation, or any serious medical disorder that would interfere with patients' safety.
7. Patients with only locally or regionally confined disease without evidence of metastatic disease.

As of January 2011, 81 patients were treated, with data for 77 patients available (Group 1=21, Group 3=34, Group 4=22). Prior therapy in the metastatic setting: a median of 2 chemotherapy lines (all patients) and 2 endocrine therapy lines (HR+ patients). A total of 58% of patients had liver metastases (Group1: 81%; Groups 3,4: 50%). Most common adverse events included: vomiting (75%; grade 3 [Group 3]: 6%), diarrhea (72%; Group 3: 6%), nausea (62%; Group 3: 5%), and asthenia (61%; Group 3: 17%). Median exposure was 1.7 months (range, 0-8.2 months), including 8 patients who received>4 months of therapy. For patients with measurable disease at baseline: Group 1, 2 (13%) had unconfirmed partial responses, and 7 (44%) patients had stable disease>4 months (SD4); Groups 3 and 4, SD4 was respectively noted in 8 (29%) and 2 (11%) patients.

Dovitinib exhibited anti-tumor activity in a pre-treated breast cancer population. Activity was observed in HR+ patients with FGFR1-amplified disease with disease stabilization observed in other subgroups. It was discovered that FGFR1 is a relevant target in breast cancer and FGFR1 amplification defines a molecular segment of dovitinib-sensitive breast cancer, as summarized from the clinical data in Table 1. Group 1 encompasses patients that are both HR+ and FGFR1 amplified. Group 3 encompasses patients that are HR+ and not amplified for FGFR1 but that could have another FGFR and/or FGF ligand amplification.

TABLE 1
Overall Response Rate for Selected Patient Groups
as Defined By Clinical Study of Example 1.

| | | |
|---|---|---|
| Group 1 | Group 3 | Group 4 |

(Data cut off Nov. 24, 2010)

| | | |
|---|---|---|
| FGFR1 | FGFR1 non- | FGFR1 non- |
| amplified | amplified | amplified |
| HR+ | HR+ | HR− |

N = (%)

| | Measurable | Measurable | Measurable |
|---|---|---|---|
| | (n = 16)* | (n = 30) | (n = 18) |
| Unconfirmed PR | 2 (12.5)¹ | — | — |
| SD | 7 (43.8) | 14 (46.7) | 4 (22.2) |
| SD ≧14 wks | 5 (31.3) | 8 (26.7) | 2 (11.1) |
| PD | 4 (25.0) | 9 (30.0) | 6 (33.3) |
| Unknown | 3 (18.8) | 7 (23.3) | 8 (44.4) |

### EXAMPLE 2

### Clinical Study to Test Efficacy of an FGFR1 Inhibitor (Dovitinib) in Patients with FGFR1-Amplified Breast Cancers

After analyzing the results of the clinical study of dovitinib from patient groups 1, 3 and 4, exploratory analyses were also performed to further evaluate clinical responses in patients with tumors bearing additional gene amplifications. Amplifications of FGF ligands (FGF3, FGF4, FGF19) as well as FGFR2 gene amplification were also performed as pre-defined in a protocol. The protocol describes methods used for copy number analysis for FGFR1 and FGF3 gene using ABI's pre-designed TaqMan™ copy number assays.

Method Summary

TaqMan® Copy Number Assays for FGFR1 and FGF3 were ordered from Applied Biosystems and are run together with a TaqMan® Copy Number Reference Assay in a duplex real-time Polymerase Chain Reaction (PCR). The Copy Number Assay detects the target gene of interest (FGFR1 or FGF3 in this case) and the Reference Assay (RNase P) detects a sequence that is known to be present in two copies in a diploid genome. This method of relative quantitation is used to determine the relative copy number of the target of interest in a genomic DNA sample, normalized to the copy number of the reference gene.

Each TaqMan® Copy Number Assay contains two unlabeled primers for amplifying and one TaqMan® MGB probe for detecting the target sequence of interest. The probe has FAM™ reporter dye attached to the 5′end and a nonfluorescent quencher (NFQ) and a Minor Groove Binder (MGB), attached to the 3′ end. MGBs increase the melting temperature (Tm) without increasing probe length.

The TaqMan® Copy Number Reference Assay contains two unlabeled primers for amplifying and one TaqMan® MGB probe for detecting RNaseP gene. The probe has VIC™ reporter dye attached to the 5′end and TAMRA™ quencher, attached to the 3′ end.

TaqMan Real-time PCR assay was performed for gene of interest (FGFR1 or FGF3) and RNase P in a duplex PCR protocol provided by ABI on BioRad™ CFX96 real time PCR instrument. Copy number calculation was based on AACt values of gene of interest (FGFR1 or FGF3) versus RNase P for unknown sample versus a normal control DNA. The normal DNA control is a commercially available genomic DNA sample that has diploid genome.

Reagents

| Reagent | Supplier/Manufacturer |
|---|---|
| Molecular Grade Water | Various |
| TaqMan Universal PCR Master Mix | Applied Biosystems |
| Hs-02109929 FGFR1 Copy Number Assay | Applied Biosystems |
| Hs-01200530 FGF3 Copy Number Assay | Applied Biosystems |
| TaqMan Copy Number RNaseP Reference Assay | Applied Biosystems |

Protocol
1. A master mix was prepared using the volumes listed below, taking into account a 10% overage for pipetting errors, and by thoroughly mixing each reagent before use.

| Component | 1 x Reaction |
|---|---|
| Molecular Grade Water | 3 μL |
| TaqMan Universal Master Mix | 5 μL |
| TaqMan Copy Number Assay, 20x | 0.5 μL |
| TaqMan RNase P Reference Assay, 20x | 0.5 μL |
| Total | 9 μL |

2. A volume of 9 μL of master mix was dispensed to each well of a PCR plate.
3. A volume of 1 μL of template DNA (10 ng/μl) was added to the wells. The PCR plates were tightly sealed and then briefly centrifuged to ensure master mix and sample are collected at the bottom of the well.
4. A reaction plate was loaded into a BioRadCFX96 real time PCR instrument.
5. The PCR assay in the reaction plate was performed using the run parameters below:
   95° C. 10 min followed by 40 cycles of
      95° C. 15 sec
      60° C. 60 sec
6. The PCR assay was set up and started running with CFX Manager Software according to CFX96 real time PCR detection system user manual.

Data Analysis

Data analysis was performed using gene expression mode according to CFX96 real time PCR detection system user manual.

Data in gene expression table was reviewed and assigned amplified/non-amplified status to the samples based on the following criteria:

Expression Value<2 indicates “non-amplified”

Expression Value=or>2 indicates “amplified”

The copy number of gene interest is 2x expression value.

References

CFX96 and CFX384 Real-Time PCR Detection Systems Instruction Manual. Bio-Rad Laboratories, 2008

TaqMan Copy Number Assays Protocol. Applied Biosystems. 2010

This analysis included all patients with measurable breast cancer, as determined by an adjudicator and with “known” FGF pathway genes status.

After profiling all the patients with FGF pathway genes, 18 patients were identified with known FGF amplified (either FGFR1, FGFR2 or FGF3)

Group 1=16 patients with FGFR1 amplification

Group 3=1 patient with FGF 3 and 1 patient with FGFR2 amplification

Group 4=none had FGF pathway genes amplified

Exploratory biomarker analyses revealed that top two patients with most tumor shrinkage are with both FGFR1 gene and FGF3 gene amplification, as summarized in Table 2. The patients exhibiting the most tumor shrinkage (<−20%) corresponded to FGFR1, FGFR2, or FGF3 gene amplification.

TABLE 2
Exploratory Biomarker Data in Patients with SD and PFS >100 days

| Patient | ADJUDICATED | PFS | Max tumor | FGFR1 | FGFR1 | FGFR2 | FGF3 |
|---|---|---|---|---|---|---|---|
| ID | Response* | ADJUDICATED | shrinkage | SISH | q-PCR | q-PCR | q-PCR |
| 2-10 | SD | 109 | −100.0 | 6.5 | 10 | no amp | 8 |
| 502-6 | NoPD4 | 224+ | −30.8 | 7.7 | 16 | no amp | 6 |
| 2-9 | SD | 111 | −28.2 | no amp | no amp | 4 | no amp |
| 2-11 | SD | 109 | −23.0 | no amp | 4 | no amp | 8 |
| 4-8 | SD | 131 | −20.8 | 8.3 | 6 | no amp | no amp |
| 3-11 | SD | 110 | −18.5 | no amp | no amp | no amp | 3 |
| 2-13 | NoPD4 | 111+ | −12.6 | no amp | no amp | no amp | no amp |
| 4-6 | NoPD4 | 107+ | −7.5 | 6.1 | 10 | no amp | 10 |

Adjudicated response: Clinical response assessed by an experienced radiologist who is blinded to the study
PFS adjudicated: progression free survival adjucated
NoPD4: patients with Stable Disease as best overall response and at least a second measurement of stable disease ≧14 wk from start therapy
SD: stable disease
After re-grouping patients with FGF pathway dysregulation, e.g. FGF amplification and without FGF pathway dysregulation, e.g.FGF-non amplification, a compelling clinical benefit was observed in the FGF amp group, with 2 unconfirmed partial response and 50% patients with stable disease, as summarized in Table 3, clearly demonstrating that dovitinib is more efficacious in patients with FGF pathway dysregulation, e.g. FGFR1, FGFR2, FGF3 gene amplification.

TABLE 3
Overall Response in Patients with FGF3
and/or FGFR1 or FGFR2 amplification

(data cut-off Nov. 24, 2010)

| | |
|---|---|
| FGF amp | FGF nonAmp |

N = (%)

| | Measurable | Measurable |
|---|---|---|
| | (n = 18)* | (n = 36) |
| Unconfirmed PR | 2 (11.1)¹ | — |
| SD | 9 (50) | 10 (27.8) |
| SD ≧14 wks | 7 (38.9) | 7 (19.4) |
| PD | 4 (22.2) | 13 (36.1) |
| Unknown | 3 (16.7) | 13 (36.1) |

According to table 3, the group of patients being non amplified FGF is encompassing patients being both HR+ and HR−. In the group of patients having FGF amplification, all patients are HR+.

The above results support the method of diagnostic and prognostic according to the present invention.

1. A method for diagnosing breast cancer in a subject, the method comprising the step of selecting the patient wherein the breast cancer is HR positive and detecting amplification of one or more biomarkers selected from a FGFR ligand and/or a FGFR and combinations thereof in the subject.

2. A method of treating breast cancer in a subject, the method comprising the steps of: (a) selecting the patient wherein the breast cancer is HR positive; (b) detecting the amplification presence or amount of one or more biomarkers selected from a FGFR ligand, and/or a FGFR, and combinations thereof in a subject; and (c) determining an FGFR1 inhibitor for treating the subject based on the presence or amount of the one or more biomarkers in the subject; and administering to the subject in need thereof the FGFR1 inhibitor.

3. A method for determining the prognosis of a subject having HR+ breast cancer and treated with an FGFR1 inhibitor, the method comprising the step of: detecting one or more biomarkers selected from a FGFR ligand, and/or a FGFR and combinations thereof in the subject, wherein the amplification of the one or more biomarkers is indicative of the prognosis of the subject treated with the FGFR1 inhibitor.

4. The method according to claim 1 wherein the FGFR1 inhibitor is dovitinib or a tautomer thereof.

5. The method according to claim 1, wherein the FGFR ligand biomarker is selected from the group consisting of the FGF3, FGF19, and FGF4 ligand amplification, preferably the FGF3 ligand amplification.

6. The method according to claim 1, wherein the biomarker comprises FGFR1 amplification.

7. The method according to claim 1, wherein the biomarker comprises FGFR2 amplification.

8. The method according to claim 5, wherein the number of amplifications ranges from 5-10.

9. The method according to claim 1 wherein the FGFR1 inhibitor is dovitinib or a tautomer thereof.
